(19) United States
(12) Patent Application Publication
(10) Pub. No.: US 2016/0100740 A1
(43) Pub. Date: Apr. 14, 2016

(54) System and Method for Display of Capsule Images and Associated Information

(71) Applicant: Capso Vision, Inc., Saratoga, CA (US)

(72) Inventors: Kang-Huai Wang, Saratoga, CA (US); Gordon C. Wilson, San Francisco, CA (US); Weiwen Lin, Cupertino, CA (US)

(73) Assignee: Capso Vision, Inc.

(21) Appl. No.: 14/974,126

(22) Filed: Dec. 18, 2015

Related U.S. Application Data
(62) Division of application No. 13/923,904, filed on Jun. 21, 2013.
(60) Provisional application No. 61/675,317, filed on Jul. 24, 2012.

(51) Int. Cl. A61B 1/00; A61B 1/04

(52) U.S. Cl. CPC A61B 1/0005; A61B 1/00009; A61B 1/041

(57) ABSTRACT

A method for presentation of image sequence data is disclosed. The intensity of the image sequence data is used to derive the navigation guide. Each representative data may correspond to an average pixel value, a median pixel value, a minimum value, or a maximum value of the sub-image or said one or more images. Each representative data may also comprise a term proportional to a mathematical product of adjusted color values. The series of representative data can be plotted along with highlighting an area between a curve corresponding to the series of representative data and a coordinate axis by filling the area with a highlight color. Each representative data may comprise a minimum value and a maximum value associated with the sub-image, or one or more images.

### CROSS REFERENCE

The present invention is a divisional application of U.S. patent application Ser. No. 13/923,904, filed on Jun. 21, 2013, which claims priority to U.S. Provisional Patent Application, Ser. No. 61/675,317, filed Jul. 24, 2012. The U.S. patent application and U.S. Provisional Patent Application are hereby incorporated by reference in their entireties.

### FIELD OF THE INVENTION

The present invention relates to diagnostic imaging inside the human body. In particular, the present invention relates to displaying images captured by a capsule camera system along with associated information.

### BACKGROUND

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Because of the difficulty traversing a convoluted passage, endoscopes cannot reach the majority of the small intestine and special techniques and precautions, that add cost, are required to reach the entirety of the colon. An alternative in vivo image sensor that addresses many of these problems is capsule endoscope. A camera is housed in a swallowable capsule, along with a radio transmitter for transmitting data, primarily comprising images recorded by the digital camera, to a base-station receiver or transceiver and data recorder outside the body. Another autonomous capsule camera system with on-board data storage was disclosed in the U.S. patent application Ser. No. 11/533,304, filed on Sep. 19, 2006.

For the above in vivo devices, a large amount of image data is collected during the course of its traverse through the human GI tract. For the autonomous capsule camera, the number of images collected may be as many as tens of thousands. The image data usually is viewed by medical professionals for diagnosis, analysis or other purposes. The image data is often displayed on a display device continuously and viewed video data at a certain frame rate, such as 30 frames per second. In order to help a viewer to navigate through the video sequence, various viewing controls such as fast forward, fast reverse, and pause are provided as part of user interface. Furthermore, annotation may be incorporated into the image data to help a physician to quickly locate images of interest. Due to the large amount of image data generated, it may take somewhere around from half an hour to hours to view the video sequence. While play control and annotation may help to expedite diagnostic process, it is desirable to develop other tools to further improve the viewing experience.

### SUMMARY OF THE INVENTION

The present invention discloses a method and system for presentation of image data. In one embodiment according to the present invention, the method comprises receiving image sequence data captured with an in vivo imaging device for GI tract; receiving annotation data associated with image sequence data, wherein the annotation data comprises location markers associated with GI tract parts; associating images from the image sequence data with the GI tract parts based on the location markers; generating a navigation guide for the images, wherein the navigation guide comprises a series of part representations corresponding to the images, and wherein one color is selected for each of the series of part representations; and displaying the navigation guide. The color can be selected from a set of color palettes. The color associated with each of the series of part representations can be pre-defined or determined interactively. Furthermore, a location indicator corresponding to one of the images currently being displayed can be displayed on the navigation bar, and the location indicator can also be displayed along with the image. In another embodiment of the present invention, a second navigation guide is generated and displayed, where the second navigation guide is generated from the navigation guide by zooming-in on a section of the navigation guide.

In another embodiment according to the present invention, the method for presentation of image data comprises receiving image sequence data captured with an in vivo imaging device for a gastrointestinal tract; generating a series of representative data, each representative data is derived based on a sub-image, or one or more images from the image sequence data; and displaying a summarized presentation for the image sequence data by plotting the series of representative data. One aspect of the present invention relates to the derivation of the series of representative data. In one embodiment, each representative data corresponds to an average pixel value, a median pixel value, a minimum value, or a maximum value of the sub-image or said one or more images. In another embodiment, each representative data comprises a term proportional to a mathematical product of adjusted color values, wherein each adjusted color value corresponds to a color representative value raised to a power for each of multiple color components, and wherein the color representative value is derived from pixels of each of multiple color components of the sub-image or said one or more images. The series of representative data can be plotted along with highlighting an area between a curve corresponding to the series of representative data and a coordinate axis by filling the area with a highlight color. Each representative data may comprise a minimum value and a maximum value associated with the sub-image, or one or more images.

### BRIEF DESCRIPTION OF THE DRAWINGS

FIG. 1A illustrates an example of a vertical navigation guide, where each shaded area corresponds to a GI tract part and can be displayed as a color bar on a display device.

FIG. 1B illustrates an example of a horizontal navigation guide, where each shaded area corresponds to a GI tract part and can be displayed as a color bar on a display device.

FIG. 2 illustrates an example of a two-dimensional navigation guide corresponding to GI tract parts.

FIG. 3 illustrates an example of derivation of the navigation guide from the annotations.

FIG. 4A illustrates an example of intensity profile corresponding to a series of representative data associated with a sub-image, an image or multiple images in one part of the GI tract, where the representative data is derived based on the red component.

FIG. 4B illustrates an example of intensity profile corresponding to a series of representative data associated with a sub-image, an image or multiple images in one part of the GI tract, where the representative data is derived based on R*R/G.

FIG. 4C illustrates an example of intensity profile corresponding to a series of representative data associated with a sub-image, an image or multiple images in one part of the GI tract, where the representative data correspond to clipped and normalized representative data of FIG. 4B.

FIG. 5A-FIG. 5C illustrate highlighted intensity profiles corresponding to the intensity profiles of FIG. 4A-FIG. 4C respectively.

FIG. 6A-FIG. 6C illustrate examples of intensity profile corresponding to FIG. 4A-FIG. 4C respectively based on another image sequence.

FIG. 7A-FIG. 7C illustrate highlighted intensity profiles corresponding to the intensity profiles of FIG. 6A-FIG. 6C respectively.

FIG. 8 illustrates an example of zoomed navigation guide based on the intensity profile.

FIG. 9 illustrates an exemplary display device displaying a current image indicated by an indicator on the intensity-profile based navigation guide.

FIG. 10 illustrates an example of zoomed navigation guide based on the GI tract parts.

FIG. 11 illustrates an exemplary display device displaying a current image indicated by an indicator on the GI tract-part based navigation guide.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention. Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

A capsule camera travels through the GI tract and exits from the human body via anus. During the course of travelling through the GI tract, the capsule camera will capture a large amount of images, which can be stored in an on-chip memory or transmitted to a base station through wireless link. The retrieved or received images are usually transferred to a base station for processing or viewing. The accuracy as well as efficiency of diagnosis is important. A health-care professional may prepare the captured images by adding annotations to identify an image or images of interest. The annotation is also called a bookmark in this disclosure. The bookmark usually is used to indicate an image or images that contain characteristics or features that are useful for viewing, diagnosis or analysis. For example, a bookmark may be used to indicate an image or images that depict the transition from one part of the GI tract to another part, such as the transition from cecum to ascending colon. A bookmark may be used to indicate an image or images that depict pathologic characteristics in the GI tract. A bookmark may also used to divide the image sequence into multiple sections to allow quick access to any of the sections for viewing. The annotations may be stores along with the data of captured images or stored separately.

The images captured using an in vivo device often have associated time stamps or numeric references, such as frame numbers. The images captured correspond to the scenes observed by the imaging sensor in the in vivo device while the in vivo device travels through the GI tract. Therefore, images of the sequence correspond to spatial locations where the corresponding images are captured. According to another point of view, the sequence correspond to time instances that the images are captured. As a useful viewing tool, a time bar or a navigation bar (corresponding to either time index or frame index) is often displayed on the side or bottom screen along with the image. The time scale or frame number can be displayed with the time bar and the location of the present image being displayed may be marked on the time bar. While the time bar provides an indication of the relative position of the present image in the sequence, the time bar does not provide any visual information related to the sequence contents.

A technique to present image sequence with visual summary is disclosed by Horn et al., in U.S. Pat. No. 7,636,092, where a summary presentation is displayed. The visual representation varying in color is generated in accordance with color varying along the data stream as the summary presentation. The summary representation is derived from data collected and is at least partially representative of a quantity and/or data collected. The derivation of the summary presentation may involve extensive computations. Accordingly, embodiments according to the present invention provide a graphic representation or profiles associated with the sequence, where the graphic representation or the profile requires little computations or no computation.

When an in vivo autonomous device travels through the GI tract, the device usually is admitted through the mouth and exits from the anus. Peristalsis propels the capsule device through the GI tract. The correspondence between the image sequence and different GI parts will be of interest to healthcare professionals/physicians. If a visual representation of correspondence between the image sequence and different GI parts can be displayed, a viewer can navigate easily and quickly to the portion of images of interest. The visual representation of correspondence between the image sequence and different GI parts may be in a form of graphic display correlated with the GI tract. For example, the GI tract can be represented as consecutive color bars correspond to various GI parts. The color corresponding to each GI tract part can be selected from a pre-defined set of color palette or a user may select a color according to his/her preference. The color bars can be arranged horizontally or vertically and can be displayed. Furthermore, the color bars can be displayed along with an image. The image currently being displayed can be indicated by an indicator or marker on the time bar. The indicator or marker may be displayed and moved around the time bar to allow a viewer to select an image in a part of the GI tract. The visual representation of correspondence, such as color bars, along with a current image indicator provides a useful tool for a viewer to navigate through the image sequence. Accordingly, the visual representation of correspondence is also called a navigation guide or navigation bar in this disclosure.

FIG. 1A illustrates an example of vertical navigation guide, where each shaded area corresponds to a GI tract part and can be displayed as a color bar on a display device. Accordingly, the navigation guide 110 in FIG. 1A may represent a portion of the GI tract or whole GI tract where the corresponding images are captured. Location indicator or marker 120 can be moved around to select an image to be displayed or the location indicator or marker 120 can be use to indicate an image currently being displayed. For example, the location indicator 120 can be moved to another location by dragging it with a computer mouse or other means (e.g., highlighting the marker using a designated key and moving the marker using arrow keys or other keys). The navigation guide 130 may also be arranges as a horizontal stripe as shown in FIG. 1B with position indicator or marker 140 to indicate an image being displayed. The drawings in FIGS. 1A-B are intended to illustrate one embodiment of the present invention where each GI tract part is represented by a color bar. The parts illustrated in FIGS. 1A-B may not drawn proportional to the actual scale.

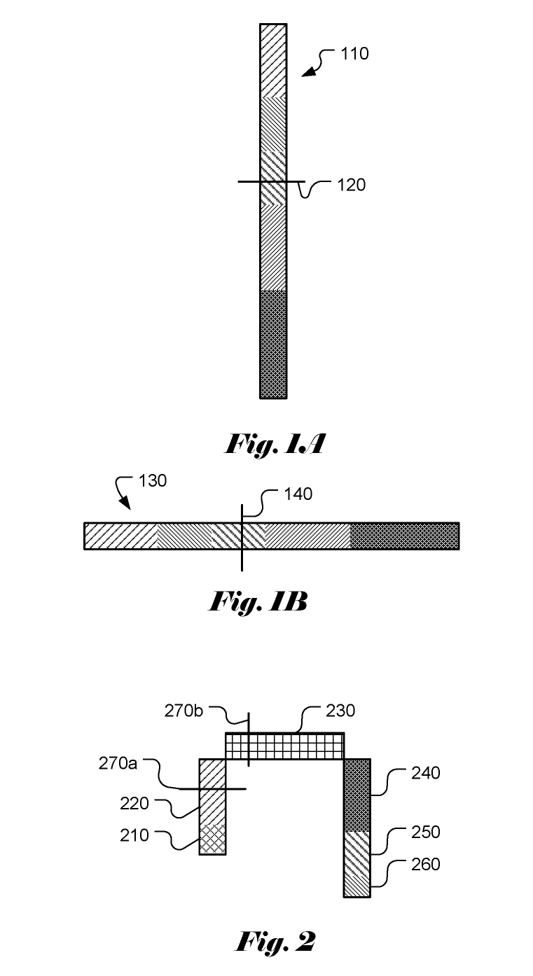

While the navigation guide can be arranged as a vertical stripe or a horizontal stripe, other arrangement may also be used. For example, the color bars may also be arranged in a way to roughly resemble the shape of GI tract inside the human body. For example, an in vivo autonomous device gathering images from the colon may use a two-dimensional navigation guide shown in FIG. 2, where the guide consists of cecum 210, ascending colon 220, transverse colon 230, descending colon 240, sigmoid colon 250 and rectum 260. Each color bar represents images captured for the corresponding GI tract part. The drawing in FIG. 2 is intended to illustrate one embodiment of the present invention where each GI tract part is represented by a color bar. The parts illustrated in FIG. 2 may not drawn proportional to the actual scale. Since the in vivo autonomous device travels in the GI tract due to paralysis, the rate of movement is usually uneven. The number of images captured in each GI tract part may not be proportional to the length of the respective GI tract part. Accordingly, the guide may not match the scale of the GI tract. If color bar length proportional to the length of the corresponding GI tract part is desirable, the image density (number of images per unit length of the color bar) can be scaled to facilitate this feature. In this case, a GI tract part where the in vivo autonomous device travels faster will have lower image density. In yet another embodiment according to the present invention, a navigation guide with continuous and curved outline closely resembling the GI tract may be used. The image sequence is mapped to the GI tract-shaped navigation guide by associating an image with a corresponding location of the GI tract. Accordingly, by moving an indicator to a place in the GI tract-shaped navigation guide, a respective image will be displayed. In FIG. 2, indicator 270a is shown on a vertical section of the navigation guide and indicator 270b is shown on the horizontal section of the navigation guide.

As mentioned before, the image data captured from a capsule camera may be associated with annotation information entered by a healthcare professional. The annotated image data can be used to generate the image navigation guide. For example, annotations may indicate first duodenum image, first jejunum image, first ileum image and first cecum image and so forth. The annotation information may have been stored along with the image data or in a separate database or file. The annotations usually also include information associated a frame number or a time record of the image sequence so that the corresponding image within the sequence can be identified. According to the time stamp or frame number, the corresponding GI tract part can be identified. According to the annotations indicating the associated part for some images, the images in the image sequence data can be mapped to the GI tract parts respectively. For example, the images from the first duodenum image to the image before the first jejunum correspond to duodenum of the GI tract. Similarly, the images from the first jejunum image to the image before the first ileum correspond to jejunum of the GI tract.

After the images corresponding to a GI tract part are identified, a part representation can be used to represent the corresponding images and a color can be applied to the corresponding part representation. The part representation forms the basis for the navigation guide and each part representation is represented as section of the navigation guide. Accordingly, in one embodiment of the present invention, the navigation guide comprises a series of part representations for the images. The color can be selected from pre-defined color palettes or a user may apply a color of his or her preference. For example, by moving a cursor over the section of the navigation guide and right clicking a mouse button, a pop-up menu may be brought up to allow a user to “select from color palette”. A set of color palettes may be display to allow a user to select one from the pre-defined color palette or to further select a custom color. In another example, by moving a cursor over the section of the navigation bar and right click a mouse button, a pop-up menu may be brought up to allow a user to select “apply default color”. In another embodiment according to the present invention, a pre-assigned color may be associated with each GI tract part. Upon identification of the GI tract part, a corresponding color may be automatically selected and/or applied. For example, a color may be pre-assigned to “duodenum”. If a section of the navigation bar is identified as “duodenum”, the pre-assigned color can be selected automatically and applied upon user's confirmation.

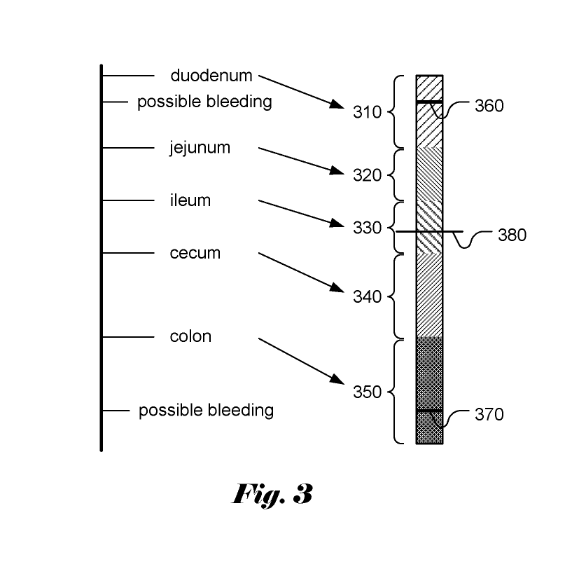

The annotations may also indicate anomaly or pathological significance. For example, one or more images may have associated annotations such as “possible bleeding” or “possible polyp”. Such images deserve special attention from a physician and are considered “high significance” images in this disclosure. An embodiment according to the present invention will highlight images with high significance. The highlight can be accomplished by assigning visually noticeable color such as a bright red color. The highlight may also be accomplished by alternating the intensity of the color, such as flashing the color at a rate of once every few seconds up to once every fractional second. Furthermore, both bright color and flashing color can be used to facilitate highlight. FIG. 3 illustrates an example of derivation of navigation guide from the annotations. The annotations designating first image or images of each part of the GI tract are used to map the images into GI tract parts (310 through 350 in FIG. 3) on the navigation guide. The annotations related to high significance are highlighted using bright color and/or flashing color (360 and 370 in FIG. 3). A location indicator 380 may be used to indicate the corresponding location in the navigation bar for the picture being displayed. In the above example, annotations associated with location markers are based on the first image of a respective part. However, other location related annotations may be used. For example, the annotations may indicate the number of images in each respective GI tract part. Furthermore, the navigation guide based on the part representation assigns a color to each part representation. Unlike the visual summary method as disclosed by Horn et al., in U.S. Pat. No. 7,636,092, where the visual representation varying in color is generated in accordance with color varying along the data stream, the present invention does not determine the color for the navigation guide in accordance with color varying along the image sequence data. Instead, the color is selected from a set of color palettes or assigned by a user.

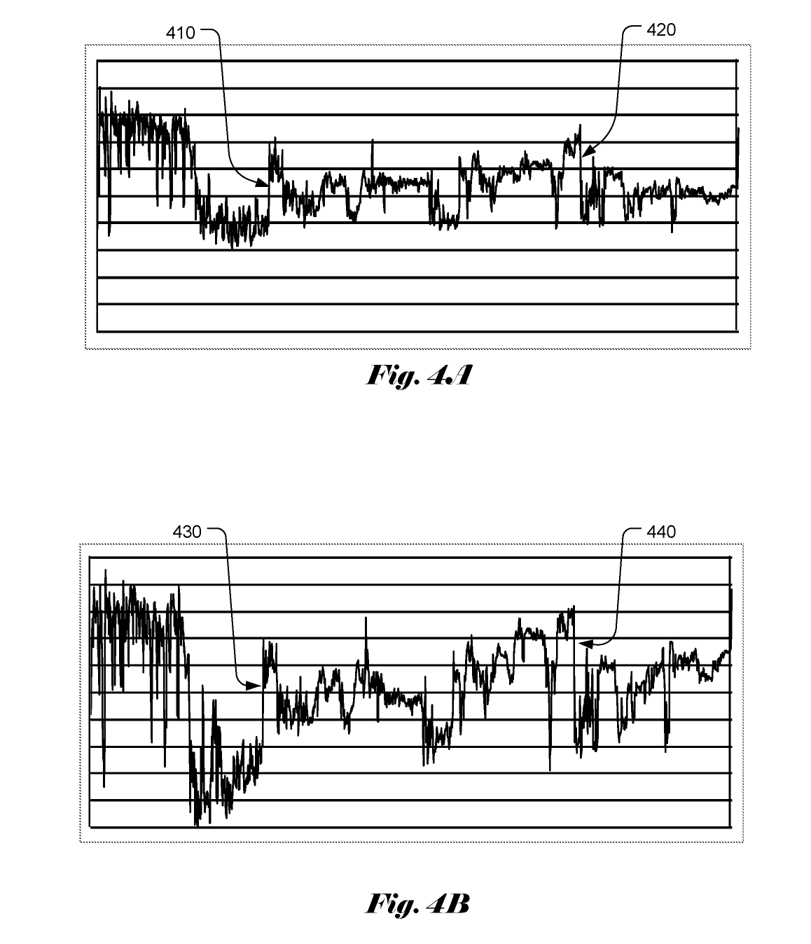

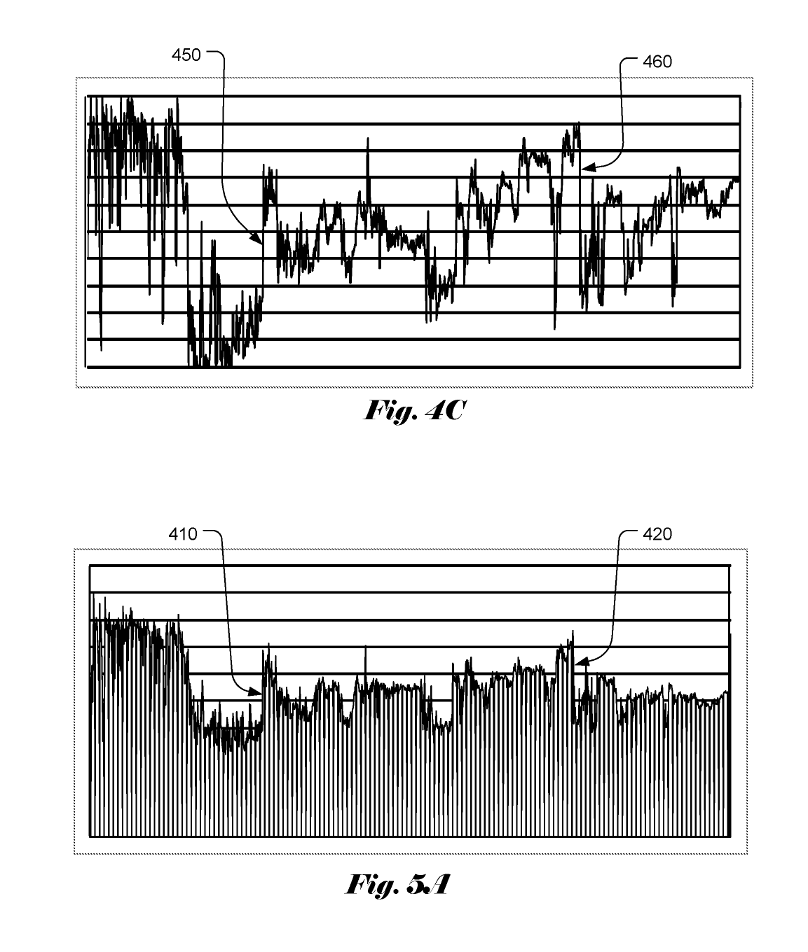

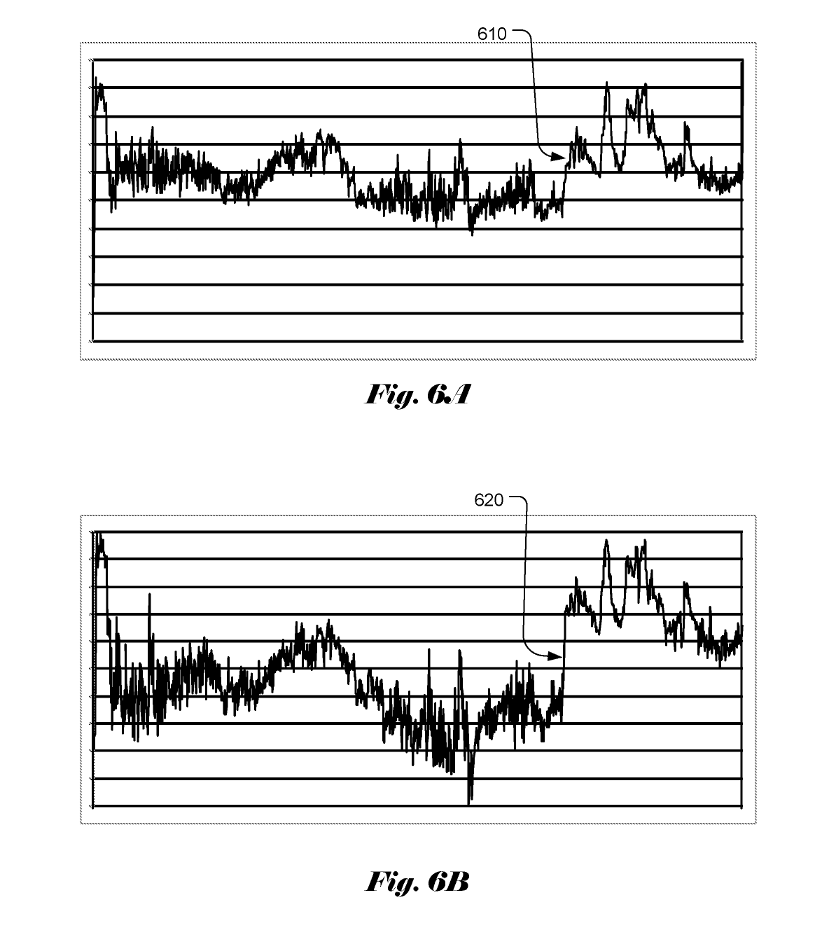

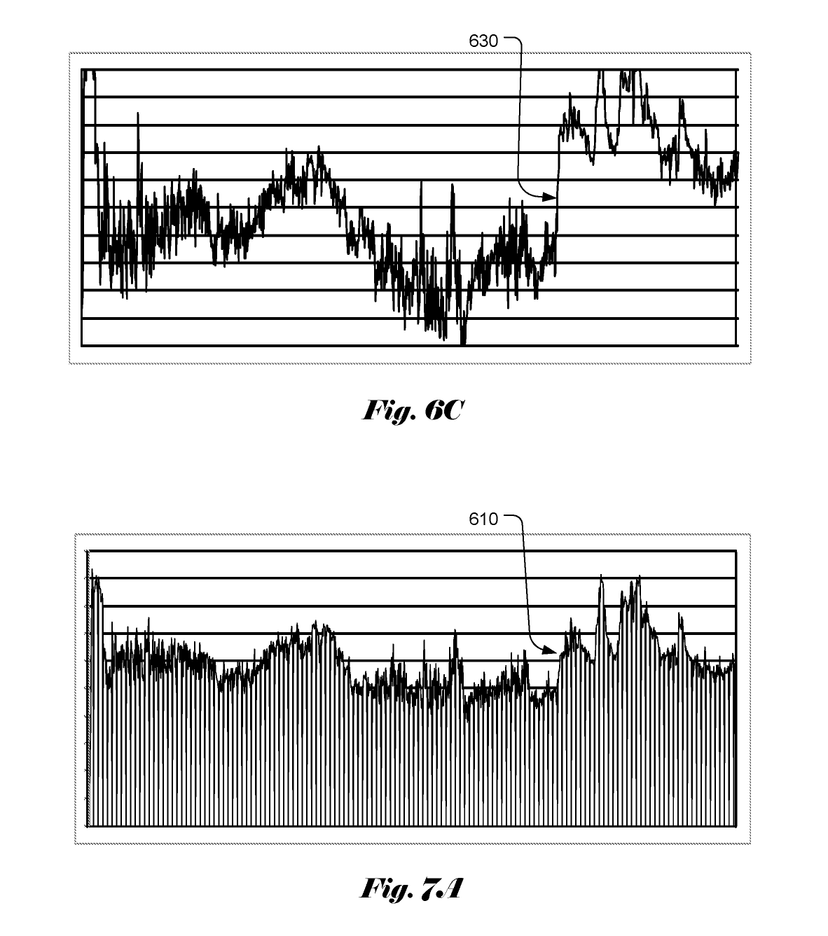

While the color-coded navigation guide provides a useful tool for a viewer to navigate around the sequence to search for images of interest, an intensity-based navigation guide also lends a useful tool for a viewer to navigate around the sequence and to spot points of interest. According to one embodiment of the present invention, image intensity profile is used as the basis for the navigation guide. FIG. 4A illustrates an example of red color profile, where each data point (i.e., each representative data) corresponds to a red intensity value averaged over a set of video data. The set of video data may correspond to a sub-image, an image or multiple images. For example, the sub-image may correspond to one or more pixel lines, one or more pixel columns, or an image area consisting of M×N pixels. Furthermore, any of these sub-images may correspond to sub-sampled video data. Due to the large quantity of images in the sequence, the number of representative data may exceed the resolution supported by the display device. In order to fit the representative data into the display area, a sub-set of the representative data may be used for display. The sub-set of representative data may be formed by decimation or averaging. For example, if the number of representative data is 20,000 while the display window only supports 1,000 data points, the sub-set of representative data can be formed by retaining one out of 20 representative data or by averaging over 20 representative data. A side by side comparison between the image sequence and the intensity profile illustrates great correlation. For example, a sharp upward transition at location 410 can be easily spotted visually. By examining the image sequence, the corresponding images demonstrate noticeable changes in image characteristics. In another example, a sharp downward transition at location 420 can be easily spotted visually and the corresponding images also demonstrate noticeable changes in image characteristics. Therefore, the intensity profile can be a useful tool to screen potential locations having clinical interest or significance. Horizontal grid lines may be displayed along with the intensity profile to serve as reference levels. FIG. 6A illustrates another example of red color profile for a second image sequence. Again, the variations appeared in the drawing illustrate high level correlation with the contents of the corresponding image sequence. A point of interest 610 is selected as shown in FIG. 6A to compare with other intensity profiles.

The red color usually is more pathologically interested than other color components. However, other primary color components may also be used to generate the intensity profile. Furthermore, a component of other color coordinate systems may also be derived from the primary colors of the image and used to generate the intensity profile. For example, the RGB primary colors may be converted to YUV or HSV (Hue Saturation Value) color coordinates.

Besides individual color components, a combination of one or more color components may reveal certain characteristics of the underlying images. For example, according to test results based on a set of capsule images captured inside the human gastrointestinal track, the ratio of red intensity to green intensity, i.e., R/G, often provides close correlation between points of interest determined based on visual inspection of the color summary representation and points of interest determined based on the R/G profile. Furthermore, two separate profiles may be combined to further enhance the capability to spot points of interest. For example, while both the red profile and the R/G profile have been shown to be useful to reveal characteristics of the underlying images, the product of R and R/G, i.e., R*R/G can further enhance the capability to spot points of interest. As another example, the product of any profile with itself also enhances the capability to spot points of interest. For example either R² or (R/G)² can be used to enhance the capability to spot points of interest.

While the exemplary combinations of profiles are provided herein, the present invention is not limit to these specific examples. In general, embodiments of the present intention may use representative data comprising a term proportional to a mathematical product of adjusted color values. Each adjusted color value corresponds to a color representative value raised to a power for each of multiple color components, and the color representative value is derived from pixels of each of multiple color components of the set of video data.

FIG. 4B illustrates an example of plotting a series of representative data corresponding to R*R/G for the same image sequence used to derive the representative data in FIG. 4A. FIG. 4B shows noticeable improvement over the representative data in FIG. 4A as evidenced by the more prominent characteristics such as transitions, valleys and peaks. For example, transitions 430 and 440 have much higher transient amplitude than the corresponding transitions 410 and 420. Similar improvement has also been noticed for the second image sequence. The series of representative data corresponding to R*R/G for the second image sequence is shown in FIG. 6B. The transition 620 in FIG. 6B corresponds to the transition 610 in FIG. 6A, and the transition 620 is much more noticeable than the transition 610. It is noted that R*R/G is mathematically equivalent to R²*G⁻¹.

While the plotting for R²*G⁻¹ shows enhanced signal characteristics, the representative data swings in a limited range. The representative data can be clipped at the high end using a high threshold. Therefore, if the representative value is larger than the high threshold, the representative value is clipped at the high threshold. Similarly, the representative data can be clipped at the low end using a low threshold. Therefore, if the representative value is smaller than the low threshold, the representative value is clipped at the low threshold. The representative data can be clipped at the high end, low end, or both high and low ends. The clipped data has a smaller dynamic range and can be normalized to restore the full range. The clipping process followed by normalization effectively serves the purpose of vertical zoom-in for mid-range data. FIG. 4C illustrates an example of clipped and normalized plotting for the representative data of FIG. 4B, where the high threshold corresponds to 90% of the full range of the representative data and the low threshold corresponds to 10% of the full range of the representative data. The transitions 450 and 460 become more noticeable than the corresponding transitions 430 and 440. FIG. 6C illustrates an example of clipped and normalized plotting for the representative data of FIG. 6B, where the high threshold corresponds to 90% of the full range of the representative data and the low threshold corresponds to 10% of the full range of the representative data. The transition 630 becomes more noticeable than the corresponding transition 620. The specific high threshold (90%) and low threshold (10%) are used for illustration purpose and should be construed as limitations of the present invention.

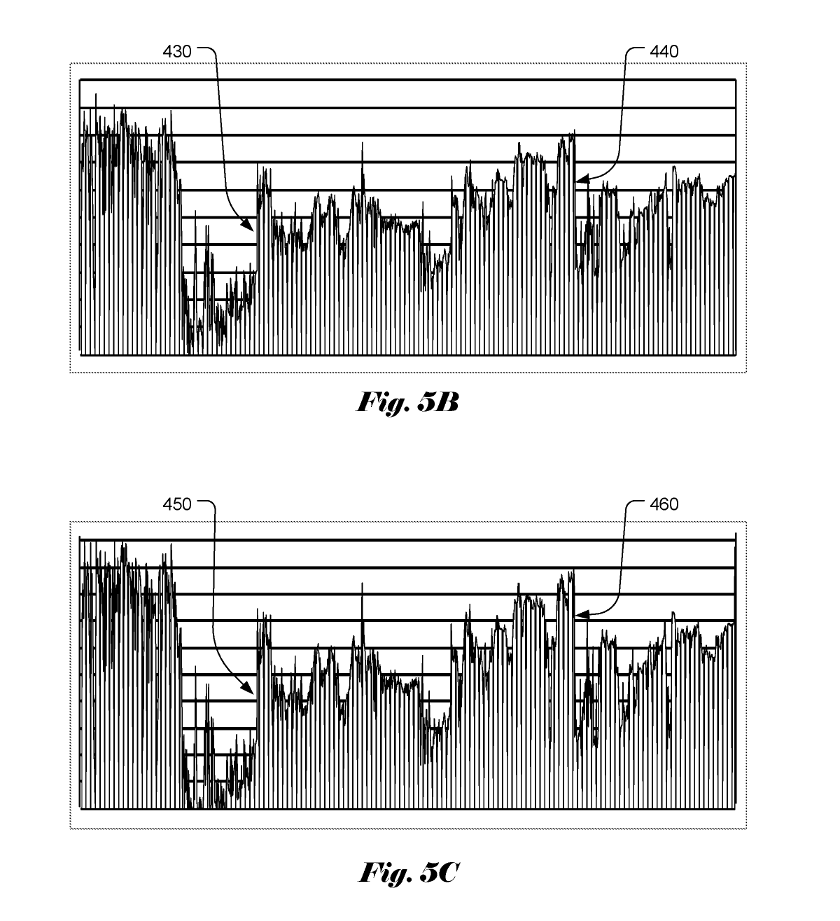

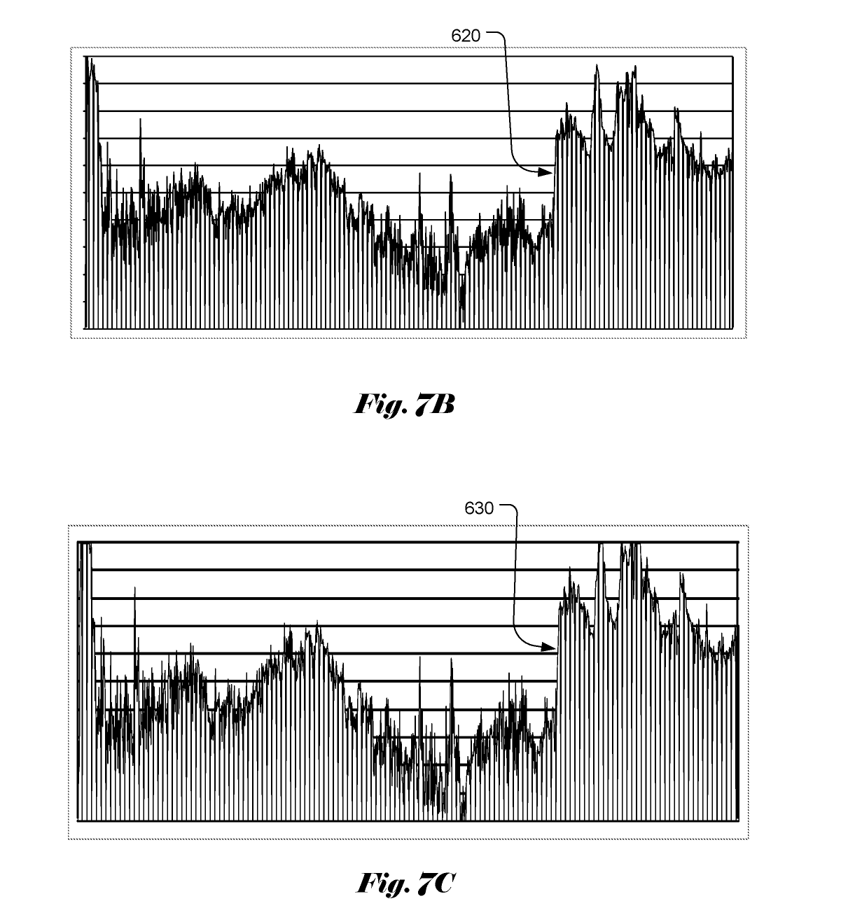

For data plotting, a highlight color filled in the area below the curve often improves data visualization. Therefore, an embodiment according to the present invention applies a high light color in the area below the curve of representative data. The highlight color may be a solid color, a mixed color, a texture, a line pattern or any shading, where the color includes a gray-level shading and black color. FIG. 5A through FIG. 5C illustrate examples of plotting representative data with highlighted color corresponding to FIG. 4A through FIG. 4C respectively, where the line patterned area indicates the highlight color. Similarly, FIG. 7A through FIG. 7C illustrate examples of plotting representative data with highlighted color corresponding to FIG. 6A through FIG. 6C respectively.

The navigation guide is a very condensed representation of the image sequence. A typical display device may have about two thousand pixels or less horizontally and a high-resolution display device may have twice as many pixels horizontally. Nevertheless, a typical image sequence captured by a capsule camera may consist of thousands or tens of thousands of images. Therefore, each data point of the navigation guide may have to represent multiple images. As mentioned previously, the representative data to be displayed may be derived by decimating or averaging the representative data. Besides averaging, the median function may also be applied to select a representative data to be displayed among multiple representative data.

While the representative data may be derived from each sub-image based on the average intensity of the sub-image, other means for deriving the representative data may also be used. For example, the intensity profile may be derived based on the minimum and/or maximum of the sub-image. Both the minimum and maximum of the samples can be easily determined and the derivation requires very low level computations. When the representative data include the minimum and maximum values for each sub-image, the intensity profile may be plotted by a range from the minimum to the maximum. For example, each data point in the displayed intensity profile may be represented by a vertical bar associated with the minimum and the maximum values. The minimum-maximum based representative data may reveal different characteristics of the underlying images from the representative data associated with average. For example, the minimum-maximum based representative data may reveal bleeding in the underlying images since the bleeding cause a high peak value of representative data (such as R*R/G) if the sub-image is comparable to the bleeding area. In addition, the average based intensity profile may be combined with the minimum-maximum based intensity profile. For example, the average based intensity profile may be displayed in a difference color by overlaying the intensity profile with the minimum-maximum based intensity profile.

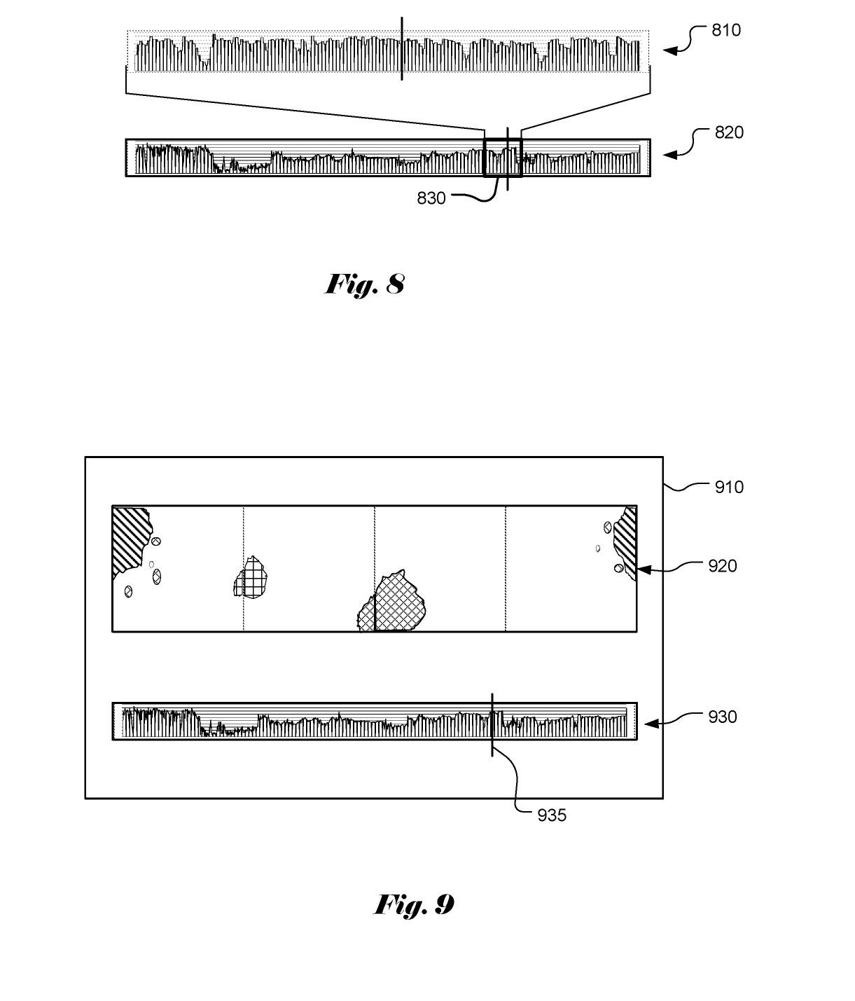

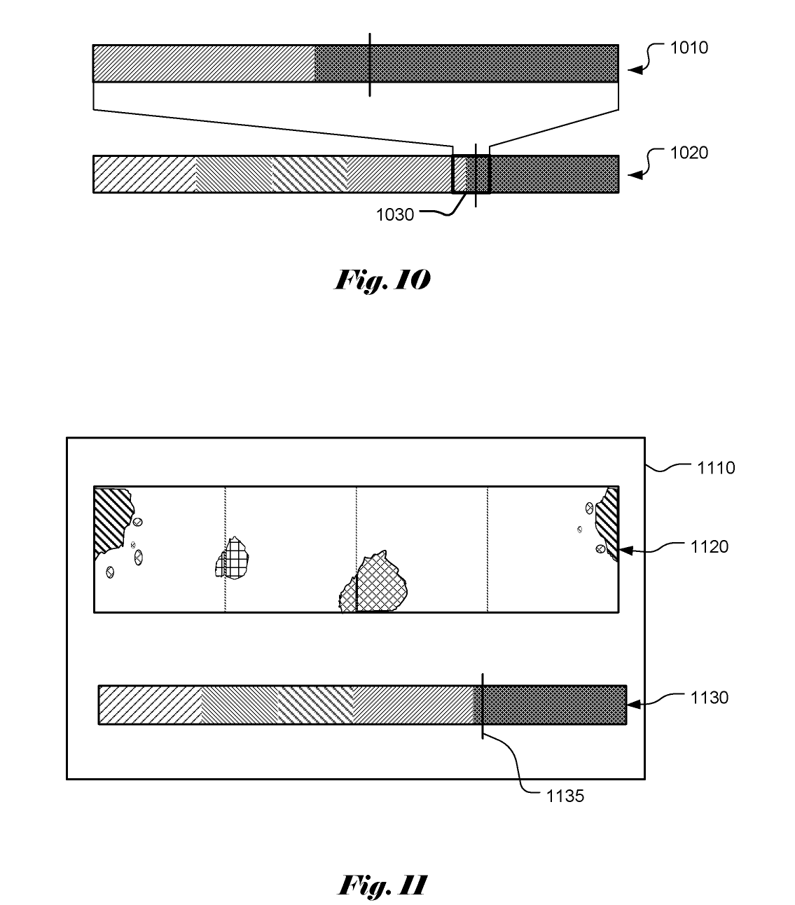

As mentioned before, the number of images in a sequence is usually much larger than the horizontal resolution of the display device. Therefore, each data point of the navigation guide may correspond to multiple images. It is desirable to allow a viewer to zoom in on the navigation guide so that the navigation guide may reveal more detailed profile associated with images. FIG. 8 illustrates an example of a zoomed navigation guide 810 from the original guide 810, where a 10× zoom is illustrated as an example. The zoomed navigation guide provides the viewer with finer navigation control so that the image or images of interest can be located. The section of the original navigation guide corresponding to the zoomed navigation guide is indicated by box 830. The zoomed navigation guide is also useful for the navigation guide based on GI tract parts. FIG. 10 illustrates an example of a zoomed navigation guide 1010 along with the original navigation guide 1020. The section of the original navigation guide corresponding to the zoomed navigation guide is indicated by box 1030. The zoom feature may be activated by an indication from user interface such as pressing a key, a button, or a combination of keys. Also a computer pointing device such as a mouse with one or more buttons may be used for a user to active the zoom feature and enter a desired zoom factor.

In one embodiment according to the present invention, the navigation guide can be displayed along with an image from the image sequence data, where an indicator or a marker associated with the navigation guide to indicate the location of the image currently being displayed. FIG. 9 illustrates a display device 910 displaying a current image 920 indicated by indicator 935 on intensity-profile based navigation guide 930. FIG. 11 illustrates a similar arrangement as FIG. 9 by using a GI parts based navigation guide 1130, where a display device 1110 displaying a current image 1120 indicated by indicator 1135.

An embodiment of the present invention to perform the steps disclosed in this application can be based on an application specific integrated circuit (ASIC), a microcontroller, or a hardware-based processor. An embodiment of the present invention may also be program codes to be executed on a Digital Signal Processor (DSP) to perform the processing described herein. The invention may also involve a number of functions to be performed by a computer processor, a digital signal processor, a microprocessor, or field programmable gate array (FPGA). These processors can be configured to perform particular tasks according to the invention, by executing machine-readable software code or firmware code that defines the particular methods embodied by the invention. The software code or firmware codes may be developed in different programming languages and different format or style. The software code may also be compiled for different target platform. However, different code formats, styles and languages of software codes and other means of configuring code to perform the tasks in accordance with the invention will not depart from the spirit and scope of the invention.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for presentation of image sequence data, the method comprising:
receiving a sequence of images captured with an in vivo imaging device for a gastrointestinal tract;
generating a series of representative data values, each representative data value is derived based on a sub-image, or on one or more images from the image sequence data; and
displaying a summarized presentation for the sequence of images by plotting the series of representative data values.

2. The method of claim 1, wherein the sequence of images is captured in a chronological order when the in vivo imaging device travels through the gastrointestinal tract and the series of representative data values are plotted in a spatial order corresponding to the chronological order associated with corresponding sub-images or images.

3. The method of claim 1 wherein each image of the sequence of images corresponds to spatial locations in the gastrointestinal tract where a corresponding image is captured.

4. The method of claim 1 wherein the summarized presentation represents at least a portion of the gastrointestinal tract where corresponding images are captured.

5. The method of claim 1, wherein each representative data value corresponds to an average pixel value of the sub-image or said one or more images.

6. The method of claim 1, wherein each representative data value corresponds to a median pixel value of the sub-image or said one or more images.

7. The method of claim 1, wherein each representative data value corresponds to a minimum value or a maximum value of the sub-image or said one or more images.

8. The method of claim 1, wherein each representative data value corresponds to a number mathematically derived from color value of pixels of the sub-image or of said one or more images.

9. The method of claim 1, wherein each representative data value comprises a term proportional to a mathematical product of adjusted color values, wherein each adjusted color value corresponds to a color representative value raised to a power for each of multiple color components, and wherein the color representative value is derived from pixels of each of multiple color components of the sub-image or said one or more images.

10. The method of claim 9, wherein the power corresponds to a positive real number, a negative real number, or a 0.

11. The method of claim 9, wherein the multiple color components correspond to red color, green color and blue color, and wherein the power for the red color is 1, the power for the green color is 0, and the power for the blue color is 0.

12. The method of claim 9, wherein the multiple color components correspond to red color, green color and blue color, and wherein the power for the red color is 2, the power for the green color is −1, and the power for the blue color is 0.

13. The method of claim 1, wherein the series of representative data values are modified to generate a series of modified representative data values by subtracting an offset from the series of representative data values.

14. The method of claim 1, wherein the series of representative data values are modified to generate a series of modified representative data values by clipping the series of representative data values exceeding a high threshold to the high threshold.

15. The method of claim 1, wherein the series of representative data values are modified to generate a series of modified representative data values by clipping the series of representative data values smaller than a low threshold to the low threshold.

16. The method of claim 15, wherein the series of modified representative data values are plotted by stretching the series of modified representative data values to a normalized range.

17. The method of claim 1, wherein said plotting the series of representative data values highlights an area between a curve corresponding to the series of representative data values and a coordinate axis by filling the area with a highlight color.

18. The method of claim 1, wherein each representative data value comprises a minimum value and a maximum value associated with the sub-image or said one or more images, and wherein said plotting the series of representative data values fills between the minimum value and the maximum value with a highlight color for each representative data value.

19. A system for presentation of image sequence data, the system comprising:
a processor configured to:
receive image sequence data captured with an in vivo imaging device for a gastrointestinal tract;
generate a series of representative data values, each representative data value is derived based on a sub-image, or one or more images from the image sequence data; and
display a summarized presentation for the image sequence data by plotting the series of representative data values.
